(19) United States
(12) Patent Application Publication
(10) Pub. No.: US 2018/0115841 A1
(43) Pub. Date: Apr. 26, 2018

(54) SYSTEM AND METHOD FOR REMOTE HEARING AID ADJUSTMENT AND HEARING TESTING BY A HEARING HEALTH PROFESSIONAL

(71) Applicant: III HOLDINGS 4, LLC, Wilmington, DE (US)

(72) Inventors: Russell J. Apfel, Austin, TX (US); Gregory Allen Bryant, Georgetown, TX (US); Andrew Lawrence Eisenberg, Austin, TX (US); Frederick Charles Neumeyer, Austin, TX (US); Gregory Charles Yancey, Austin, TX (US); Harold S. Mindlin, II, Austin, TX (US); Donald Coplin, Austin, TX (US)

(21) Appl. No.: 15/847,837

(22) Filed: Dec. 19, 2017

Related U.S. Application Data
(63) Continuation of application No. 13/708,719, filed on Dec. 7, 2012.
(60) Provisional application No. 61/583,911, filed on Jan. 6, 2012.

(51) Int. Cl. H04R 25/00; A61B 5/12; A61B 5/00

(52) U.S. Cl. CPC H04R 25/558; A61B 5/121; A61B 5/0022; H04R 2225/55; H04R 25/70

(57) ABSTRACT

A system for establishing a communication to communicate with a hearing health professional, providing data related to hearing aid settings to the hearing health professional, receiving a hearing aid profile in response to providing the data, and providing the hearing aid profile to a hearing aid through a second communication link.

### CROSS-REFERENCE TO RELATED APPLICATION(S)

This application is a continuation of U.S. application Ser. No. 13/708,719, filed on Dec. 7, 2012, which claims priority to Provisional Application No. 61/583,911, filed on Jan. 6, 2012 and entitled “SYSTEM AND METHOD FOR REMOTE HEARING AID ADJUSTMENT AND HEARING TESTING BY A HEARING HEALTH PROFESSIONAL,” which are incorporated herein b reference in their entireties.

### FIELD

This disclosure relates generally to system and methods of performing a hearing examination, and more particularly to systems and methods of remote hearing aid tuning by a hearing health professional.

### BACKGROUND

Hearing deficiencies can range from partial to complete hearing loss. Often, an individual's hearing ability varies across the range of audible frequencies, and many individuals have hearing impairments with respect to only select acoustic frequencies. For example, an individual's, hearing loss may be greater at higher frequencies than at lower frequencies within the audible range, or vice versa.

Hearing aids have been developed to alleviate the effects of hearing losses in individuals. Conventionally, hearing aids are configurable to amplify or otherwise modulate sounds to compensate for the particular hearing impairment of a patient. Conventionally, each hearing aid is tuned by a hearing health professional to compensate for the unique variations of the individuals hearing loss in each ear. A hearing health professional takes measurements using calibrated and specialized equipment to assess an individuals hearing capabilities and then adjusts the hearing aid parameters based on the measurements. In some instances, the hearing health professional may create multiple hearing aid profiles by adjusting the hearing aid parameters differently for use in different sound environments. Such hearing profiles include frequency and amplitude adjustments that can be applied to sound-related signals to compensate for a particular user's hearing deficiencies, to filter frequencies and/or to reduce the volume in certain acoustic environments.

However, a user's hearing may change over time. Accordingly, the measurements taken by the hearing health professional may need to be adjusted from time to time. To have adjustments made, the user may need to make multiple visits to the hearing health professional during a period of time after the initial fitting.

### BRIEF DESCRIPTION OF THE DRAWINGS

FIG. 1 is a block diagram of an embodiment of a system including a hearing aid, a computing device, and a customer service data system adapted to provide real-time hearing testing or hearing aid tuning by a hearing health professional.

FIG. 2 is a block diagram of an embodiment of the system of FIG. 1 including customer service system and a database,

FIG. 3 is a block diagram of an embodiment of the system including a computing device and a customer service data system adapted to provide real-time hearing testing.

FIG. 4 is a flow diagram of an embodiment of a method of receiving hearing aid updates from a customer service data system.

FIG. 5 is a flow diagram of an embodiment of a method of providing hearing aid updates to a hearing aid user.

In the following description, the use of the same reference numerals in different drawings indicates similar or identical items.

### DETAILED DESCRIPTION OF ILLUSTRATIVE EMBODIMENTS

Embodiments of a system and methods for remote testing of hearing loss and tuning of hearing aids by a hearing health professional are described below that allow the hearing health professional to make remote measurements and adjustments to the user's hearing aid settings in real-time (or near real-time), such as while the user is located in the problematic acoustic environment. In an example, a computing device includes a transceiver configured to communicate wirelessly with a hearing aid to provide data to the hearing aid, such as an updated hearing aid profile. As used herein, the term “hearing aid profile” refers to a collection of parameters and coefficients that can be applied by a processor of the hearing aid to process sounds to produce a modulated output signal that compensates for the user's hearing impairment. The computing device can be a phone, personal digital assistant, or other portable electronic device, which is configurable to communicate through a network with a remote customer service data system that can be operated by a hearing health professional.

In an embodiment, the user may interact with the computing device to trigger an application that communicates with the remote customer service data system through the network and interacts with a user interface of the computing device to communicate to the remote customer service data system. The data may be a current hearing aid profile and user information, such as issues with the hearing aid and/or user preferences.

In another embodiment, the user may interact with the computing device to trigger an application that communicates with the remote customer service data system through the network to provide a hearing test, a hearing screening, a hearing evaluation, to return a feedback canceler, or provide a real ear insertion gain test using the hearing aid with or without the aid of an audiologist.

In response to receiving the data from the computing device, the remote customer service data system communicates with a hearing health professional. In one example, the remote customer service data system communicates the data to a computing device of the hearing health professional via a secure connection. In another example, the hearing health professional may then update the hearing aid profile and provide the updated hearing aid profile to the computing device of the user for updating the hearing aid. In another example, the hearing health professional may direct a hearing evaluation or hearing test being taken by the user and at the end of the test allow the user to order a hearing aid that the hearing health professional can program based on the results of the directed hearing evaluation or hearing test. In this way the user is no longer required to make initial visit or even a return visit to the hearing health professional to have his or her hearing aids professionally programmed.

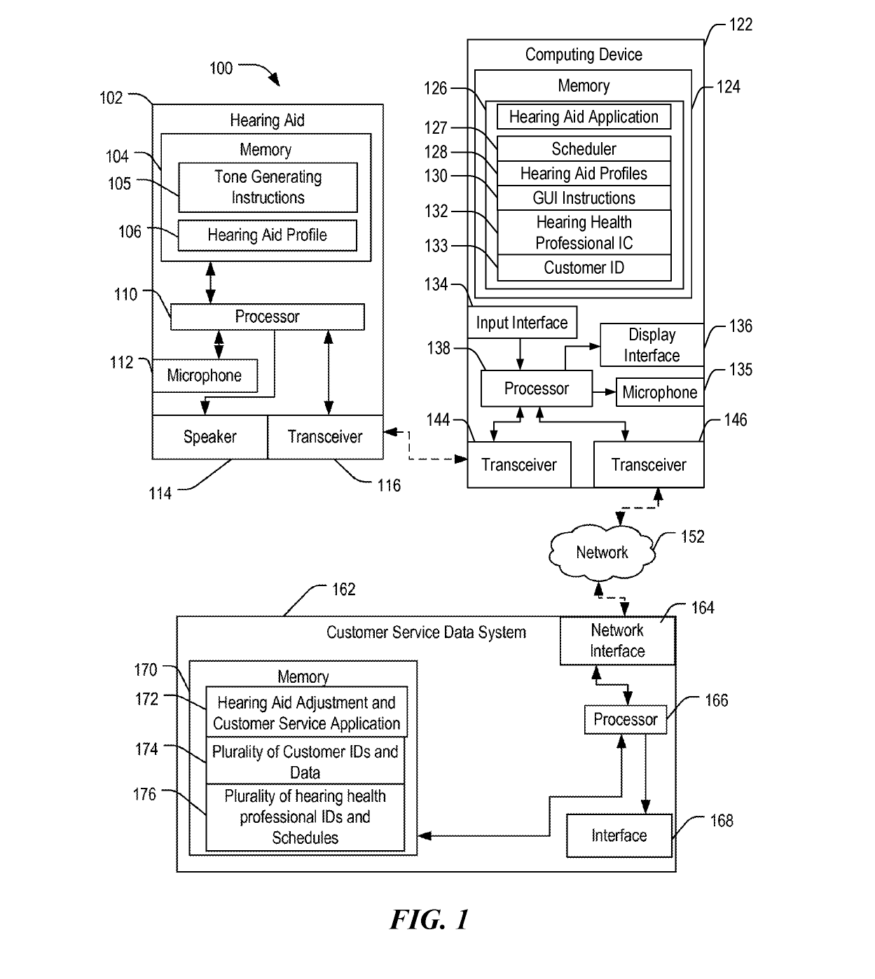

In the examples, the user selects an option by interacting with a user interface of a computing device. In some instances, the user interface is a touch screen and the user-selectable option represents an input mechanism (such as a selectable link or button) by which the user may initiate communication with a hearing health professional. In response to the user selecting the option, the computing device establishes a voice, text, or video communication link with the hearing health professional to allow the hearing health professional to communicate with the user, discuss the user's issue, and, through the remote customer service data system, establish a secure connection to the computing device, which allows the hearing health professional to view, edit, and/or provide adjustments, such as a new hearing aid profile in addition to conducting hearing evaluations via the computing device. The hearing health professional may adjust the hearing aid profiles iteratively through the remote customer service data system until the user is comfortable with the fitting. An example of a system to facilitate remote adjustment is described below with respect to FIG. 1.

FIG. 1 is a block diagram of an embodiment of a system 100 including a hearing aid 102 in communication with a computing device 122, and a customer service data system 162 also in communication with computing device 122. System 100 adapted to provide real-time hearing testing or hearing aid tuning by a hearing health professional, for example through a network 152.

Hearing aid 102 includes a processor 110 coupled to one or more computer-readable storage media 104. A computer-readable storage media may be an example of non-transitory computer storage media and may include volatile and nonvolatile memory and/or removable and non-removable media implemented in any type of technology for storage of information such as computer-readable instructions, data structures, program modules or other data. Such computer-readable media includes, but is not limited to, RAM, ROM, EEPROM, flash memory or other computer-readable media technology, CD-ROM, digital versatile disks (DVD) or other optical storage, magnetic cassettes, magnetic tape, solid state storage, magnetic disk storage, RAID storage systems, storage arrays, network attached storage, storage area networks, cloud storage, or any other medium that can be used to store information and which can be accessed by the processor 104 directly or through another computing device. Accordingly, the computer-readable storage media 104 may be computer-readable media able to maintain instructions, modules or components executable by the processor 110.

Computer-readable storage, media 104 stores processor-executable sound shaping instructions, such one or more hearing aid profiles 106 and tone generating instructions 105. The tone generating instructions 105 may include tones, verbal instructions, audio queues, words, speech parts, sounds, music, bone-conduction, vibrations, or other audible indicators used for the testing of human hearing. Hearing aid 102 further includes a microphone 112 coupled to processor 110 and adapted to receive environmental noise or sounds and to convert the sounds into electrical signals. Microphone 112 provides the electrical signals to processor 110, which processes the electrical signals according to a currently selected hearing aid profile to produce a shaped output signal. In this example, microphone 112 includes an analog-to-digital converter. Processor 110 provides shaped output signal to a speaker 114, which reproduces the modulated output signal as an audible sound at or within the user's ear canal. Hearing aid 102 further includes a transceiver 116, which is configured to communicate with computing device 122 through a communication channel.

Computing device 122 includes a processor capable of executing instructions, including but not limited to a personal digital assistant (PDA), smart phone, portable computer, tablet computer, or mobile communication device (such as a cell phone or smart phone). One representative embodiment of computing device 122 is the Apple iPhone®, which is commercially available from Apple, Inc. of Cupertino, Calif. Another representative embodiment of computing device 122 is the Blackberry® phone, available from Research In Motion Limited of Waterloo, Ontario. Other types of mobile computing devices can also be used, such as a device utilizing the Android® operating system.

Computing device 122 includes a computer-readable storage media 124, and a processor 138 coupled to computer-readable storage media 124. Computer-readable storage media 124 stores a plurality of instructions including a hearing aid application 126. Hearing aid application 126 is configured to be executed by processor 138, and includes a graphical user interface (GUI) instruction 130, a scheduler 127, a plurality of hearing, aid profiles 128, and optionally a hearing health professional identifier (ID) 132 and a customer identifier (ID) 133.

Processor 138 is further coupled to a transceiver 144, a network interface 146, a microphone 135, a display interface 136, which can display information to a user, and to an input interface 134, which is configured to receive user input. In some embodiments, a touch screen display may be used, in which case display interface 136 and input interface 134 may be combined.

Transceiver 144 is configured to communicate with hearing aid 102 through the communication channel. In an example, transceiver 144 can be a radio frequency transceiver configured to send and receive radio frequency signals, such as short range wireless signals, including Bluetooth) protocol signals, IEEE 802.11 family protocol signals, or other standard or proprietary wireless protocol signals. In some instances, the communication channel can be a Bluetooth® communication channel.

Computing device 122 further includes a network interface 146 configured to communicate data and/or audio with customer service data system 162 through a network 152, such as a Public Switched Telephone Network (PSTN), a cellular and/or digital phone network, the Internet, another type of network, or any combination thereof Network interface 146 makes it possible for various parameters associated with acoustic environments to be communicated between computing device 122 and customer service data system 162.

Customer service data system 162 includes a processor 166 coupled to a network interface 164 that is communicatively coupled to network 152. Processor 166 is further coupled to an interface 168 for communicating and receiving data from a customer service representative or hearing health professional. Processor 166 is further coupled to a computer-readable storage media 170, which stores hearing aid adjustment and customer service application 172, customer ID and data 174, a plurality of hearing health professional IDs and schedules 176, and tone generation instructions 178. Customer ID and data 174 includes a unique identifier that is linked to a particular customer and a computing device 122 known to belong to a particular customer, and customer data including hearing aid profiles, hearing loss profile, adjustment records, hearing aid make and model, and other customer service data. Tone generating instructions 178 may be accessed by a hearing health professional via the user interact 168 to direct a hearing test.

In operation microphone 112 converts environmental noise into electrical signals and provides the electrical signals to processor 110, which shapes the electrical signals according to hearing aid profile 106 to produce a modulated (modified) output signal that is customized to compensate the user's particular hearing deficiencies and optionally for the particular acoustic environment. Processor 110 provides the modulated output signal to speaker 114 which reprocess the modulated output signal as sound.

Hearing aid 102 is in communication with computing device 122 via transceivers 116 and 144. A user is able to swap hearing aid profile 106 with any of hearing aid profiles 128 and to modify the settings of hearing aid profiles 1.28 by utilizing hearing aid application 126. The user is also able to communication with a hearing health professional using computing device 122 and hearing aid application 126 via network interface 146. The hearing health professional is able to make adjustments and test the users hearing when a connection is established.

In one example, the user activates hearing aid application 126 on computing device 122, which displays a GUI generated by GUI instructions 130 on display interface 132. Using the input interface 134 and the GUI the user is able to contact a hearing health professional to have the hearing health professional provide hearing aid related support and counseling to the user including adjusting hearing aid profile 106. Once processor 138 receives a signal from input interface 134 indicating that the user wishes to contact a hearing health professional processor 138, processor 138 retrieves hearing health professional ID 132 and customer ID 133 from computer-readable storage media 124 and provides them to network interface 146 which contacts customer service data system 162. Processor 166 of customer service data system 162 receives the hearing health professional ID 132 and customer ID 133 from network interface 164, and in response activates hearing aid adjustment and customer service application 172 on interface 168, which corresponds to the hearing health professional represented by hearing health professional ID 132.

Hearing aid adjustment and customer service application 172 when executed by processor 166 cause processor 166 to access the customer data corresponding to customer ID 133 from the plurality of customer ID and data 174 and provide the customer data to interface 168. Processor 166 also connects computing device 122 to interface 168 such that the hearing health professional is able to communication data and audio between customer service data system 162 and computing device 122. Thus the user is able to talk to the hearing health professional while the hearing health professional makes adjustments to the hearing aid settings and or controls a hearing evaluation test.

In one particular example, the user may talk to the hearing health professional about the how their hearing aid profile 106 is currently working and ask the hearing professional to adjust it. The hearing health professional is able to access hearing aid profile 106 either through the customer data provided to the interface or by retrieving it from computing device 122. Computing device 122 either has hearing aid profile 106 stored in hearing aid profiles 128 or can access it through transceivers 144 and 116. Once the settings of hearing aid profile 106 are displayed to the hearing health professional on interface 168, the hearing health professional may adjust it using interface 168. At this time the user is currently, using hearing aid profile 106 and can describe to the hearing health professional the problem he/she is having with hearing aid profile 106 in real time. Once hearing aid profile 106 has been adjusted by the hearing health professional, customer service data system 162 provides hearing aid profile 106 to hearing aid 102 via computing, device 122. The user is thus able to evaluate the adjusted hearing aid profile 106 in the acoustic environment that the user was having issues in at the time the use was having the problems. If the adjusted hearing aid profile 106 is suitable the user can report this to the hearing health professional and the session can be terminated. If not the hearing health professional can listen to the user's additional issues with hearing aid profile 106 and re-adjust it. The user and the hearing health professional may continue this exercise until the user is happy with adjusted hearing aid profile 106.

The user's description of the problem or the adjustments to hearing aid profile 106 may not be enough to compensate for the user's issues, in which case the hearing health professional may request that the user to take a hearing evaluation or hearing test. In this case the hearing health professional using interface 168 may provide commands to hearing aid 102 via computing device 122, for processor 110 to access tone generating instructions 105 and to provide the tones to speaker 114. The hearing health professional may provide an automated hearing test, in which case processor 110 will receive a series of tones to play in sequence or may direct the test using interface 168 in which case the hearing health professional will direct processor 110 to play one tone at a time upon receiving the command from customer service data system 162. The user may respond to hearing the tones using hearing aid application 126 and input interface 134. Processor 138 receives the input from input interface 134 and provides it to customer service data system 162 through network 152. The inputs are displayed to the hearing health professional on interface 168. From the results of the hearing evaluation or hearing test the hearing health professional is able to generate a new hearing aid profile which is provided to computing device 122 and stored in hearing aid profiles 128. It should be understood, that the hearing evaluation may be taken either with or without environmental noise. Typically, hearing evaluations are taken in a quiet environment but by taking the hearing evaluation while microphone 112 and processor 110 is mixing in environmental noise with the tones may help the hearing health professional determine what the current setting issue is.

In another example, the user may have just received their hearing aid 102 and hearing aid profile 106 is not yet programed. In this example, the hearing health professional may test the user's hearing for the first time using hearing aid 102 to provide the initial hearing aid profile 106. By using hearing aid 102 to take the hearing evaluation that provides data for the original programing, the data takes into account the characteristics of microphone 112 improving the initial settings of hearing aid profile 106. The user may also utilize sound isolating headphones in conjunction with the hearing test to improve the quality of the results or microphone 112 or a microphone 135 on computing device 122 to measure the environmental noise and to correct the test to compensate or provide the environmental noise in the test report. For example, microphone 112 may run a sound spectrum analysis and record the results with the hearing test results that are provided to customer service data system 162.

In yet another example rather than contacting the hearing health professional immediately, processor 138 may display scheduler 127 on display interface 136, such that the user is able to schedule a time for the hearing health professional to contact the user. When processor 138 activates the scheduler, processor 138 provides hearing health professional ID 132 to network interface 146, which provides hearing health professional ID 132 to customer service data system 162. Customer service data system 162 is able to access a schedule for the hearing health professional corresponding to the hearing health professional ID 132. The request is received by network interface 164 and provided to processor 166 of customer service data system. In response to receiving the request, processor 166 access the plurality of hearing health professional IDs and schedules 176 and returns the schedule corresponding to the hearing health professional ID 132 to computing device 122 via network 152. Processor 138 of computing device 122 is then able to provide the schedule to display interface 136 and receive a user input corresponding to a time and a date the user would like to reserve for an appointment with the user's hearing health professional. Processor 138 then provides the time and the date back to customer service data system 162 and processor 166 is able to update the schedule in computer-readable storage media 170 to reflex the appointment. It should be understood that computing device 122 and customer service data system 162 may preform verification, conflict checking, and error checking as the appointment is scheduled between them.

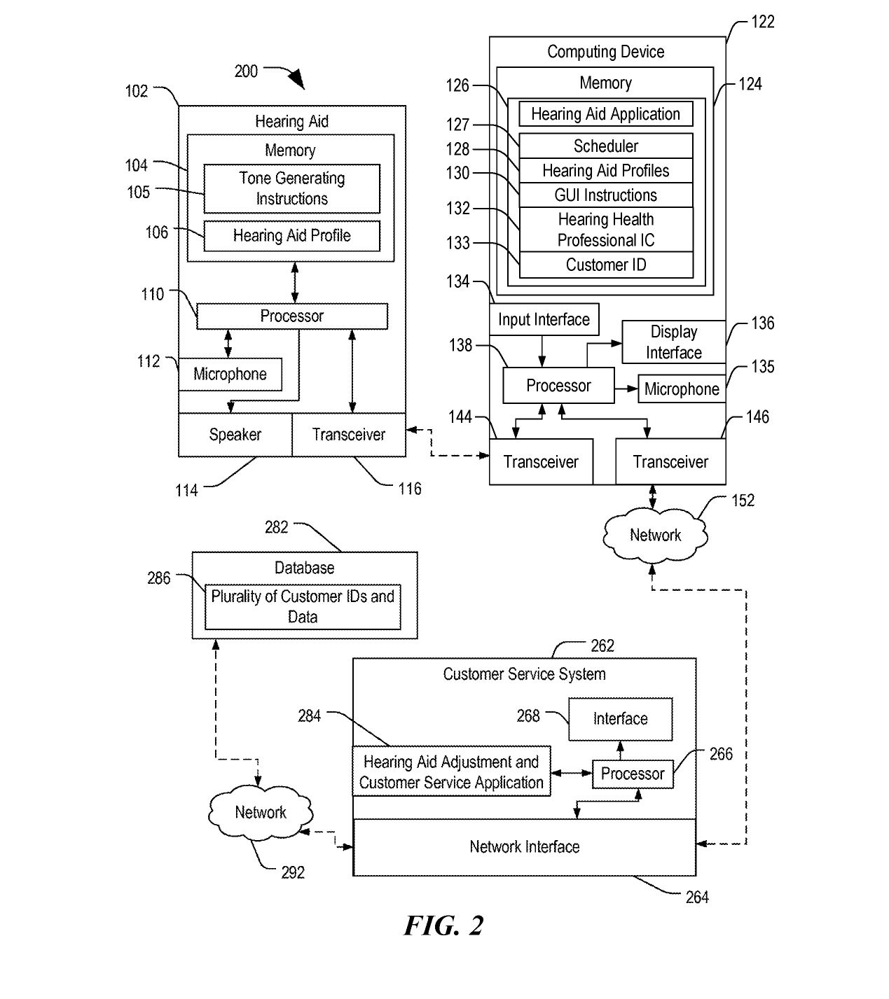

FIG. 1 shows a representative example of one possible embodiment of a customer service system for providing real time virtual hearing health support and hearing aid profile adjustment. FIG. 2 shows a second possible representative customer service system 200.

FIG. 2 is a diagram of an embodiment 200 of the system of FIG. 1 including customer service system and a database. The system includes hearing aid 102 and computing device 122 describe above in FIG. 1. The system also includes customer service system 262 and database 282. In this embodiment, customer service data system 162 is divided between a centralized database 262 and a number of independent customer service systems 262.

Customer service system 262 includes an interface 268 for providing data and receiving inputs from the hearing health professional and a network interface 264 for communication with database 282 and computing device 122 through one or more networks 152 and 292. Customer service system 262 also includes a processor 266 coupled to interface 268 and network interface 264 and hearing aid adjustment and customer service application 284, which is executable by processor 266 for providing customer support to a hearing aid user. Database 282 includes customer ID and data 286. Customer ID and data 286 includes a unique identifier that is linked to a particular customer and a computing device 122 known to belong to a particular customer, and customer data including hearing aid profiles, hearing loss profile, adjustment records, hearing aid make and model, and other customer service data. Customer data stored in database 282 is fully accessible by processor 266 through network 292.

In operation, as described above a user is able to contact a hearing health professional using hearing aid application 126 on computing device 122. The user may either use scheduler 127 to schedule an appointment with the hearing health professional or contact them immediately. Once it is time to contact the hearing health professional, processor 138 retrieves hearing health professional ID 132 from computer-readable storage media 124 and establishes a communication link with customer service system 262. Customer service system 262 is one of a plurality of customer service systems which is networked to database. 282, however, processor 138 establishes the communication link with customer service system 262, which corresponds to the customer service system that is utilized by the user's personal hearing health professional. It should be understood that if a user has never contacted a hearing health professional before about hearing aid support, hearing health professional ID 132 is set to a default and processor 138 may contact any of the plurality of customer service systems. Once the communication link is established with a customer service system and the user is able to communication with a hearing health professional, the hearing health professional ID corresponding to the hearing health professional in communication with the user is saved in hearing health professional ID,

Once the communication link between computing device 122 and customer service system 262 is established over network 152, processor 266 of customer service system 262 either request customer ID 133 from computing device 122 or received customer ID 133 when the communication link was established. Using customer ID 133 processor 266 requests customer data corresponding to customer ID 133 from database 282 through network 292 via network interfaces 264. Database 282 retrieves the customer data corresponding to customer ID 133 by matching customer ID 133 with one of the plurality of customer IDs and data 286 and provides the customer data to customer service system 262.

Processor 266 executes hearing aid adjustment and customer service application 284, which causes processor 266 to displays the customer data to the hearing health professional via interface 268 and to establish audio and/or visual communication between the hearing health professional and computing device 122. As described above, the user is able to communicate the user's issues with hearing aid 102, including hearing aid profile 106. Again the hearing health professional is able to adjust the hearing aid settings including hearing aid profile 106, provide the modified settings to hearing aid 102 through computing device 122 and activate tone generating instructions 105 to conduct a hearing evaluation using hearing aid 102.

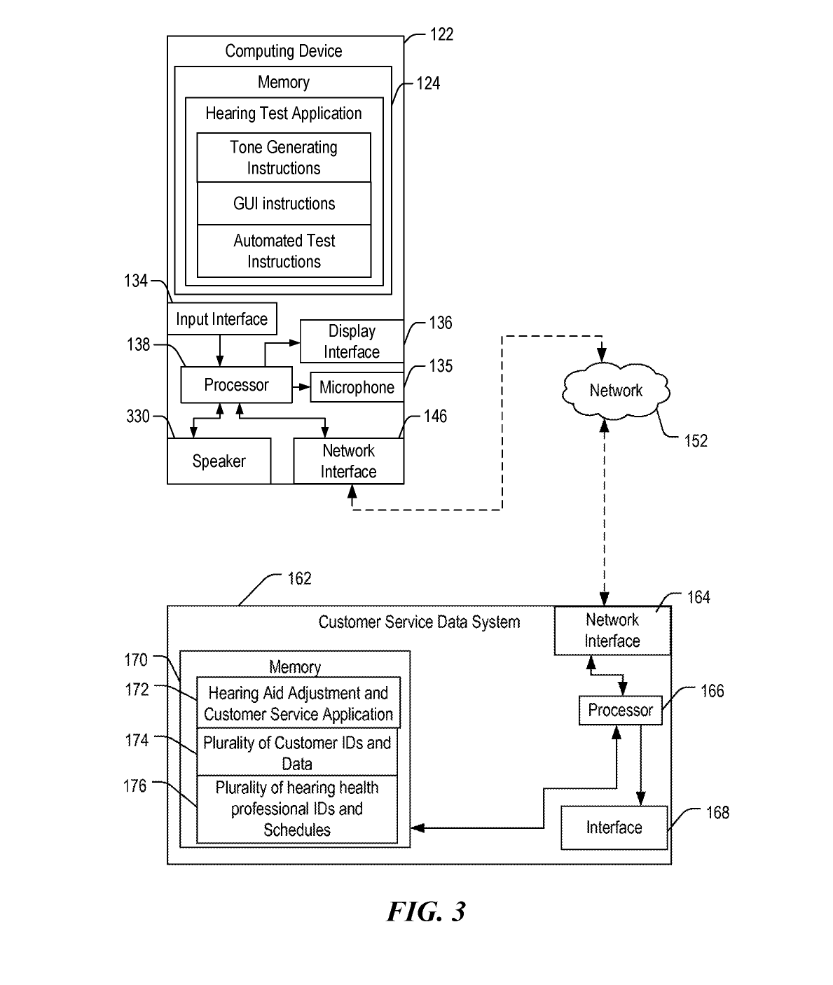

FIGS. 1 and 2 show a system for providing hearing aid adjustments and hearing evaluation testing using hearing aid 102, however, in some instances the user has not yet decided to purchase hearing aid 102 but a hearing evaluation is still required. FIG. 3 shows a system in which computing device 122 includes speaker 322 and tone generating instructions 324 for providing remote real-time hearing evaluation and hearing testing.

FIG. 3 is a block diagram of an embodiment of the system 300 including a computing device 122 and a customer service data system 162 adapted to provide real-time hearing testing. Customer service data system 162 is the same as described in FIG. 1. Computing device 122 includes computer-readable storage media 124 which stores hearing test application 322 comprising tone generating instructions 324 and GUI instructions 326. Computing device 122 also includes input interface 134, display interface 136, network interface 146, and processor 138 coupled to computer-readable storage media 124, input interface 134, display interface 136, and network interface 146. Processor 138 is also coupled to speaker 330. Speaker 330 may be headphones, an internal speaker, or an external speaker.

In an example, processor 138 executes hearing test application 322, which cause processor 138 to access GUI instructions 326 and provide hearing test instructions to display interface 136. Processor 138 also accesses automated test instructions 328 which cause processor 138 to provide tones to speaker 330 which are reproduced as sound for the user. Automated test instructions 328 cause processor to provide a tone to speaker 330 and wait for a response form the user via input interface 134. Processor 128 records the responses and lack of responses from input interface 134 as a test result and after automated test instructions 328 are completed to provide the test result to display interface 136. Once the test results are displayed to the user on display interface 136 hearing test application 322 is configured to allow the user to contact a hearing health professional to discuss the results. If the user does wish to contact the hearing health professional, processor 138 receives the input from input interface 134 and establishes a communication link with customer service data system 162 via network 152 and network interfaces 146 and 164.

Similar to the discussion of FIG. 1, customer service data system 162 allows a hearing health professional through audio and/or visual communication with the user by utilizing interface 168 and hearing aid adjustment and customer service application 172. At this time if the user is in need of a hearing aid, the user may order the hearing aid from the hearing health professional, if the user orders a hearing aid processor 138 provides the test results to customer service data system 162 and a customer ID and data entry representing the user is added to the plurality of customer IDs and data 174 and the hearing health professional is able to generate at least one hearing aid profile form the test results to store with the customer data.

In another example, the hearing health professional is able to control the hearing test by providing commands using interface 168 and hearing aid adjustment and customer service application 172 to computing device 122. In this example rather than executing automated test instructions 328, processor 138 of computing device 122, executes the commands and in response provides tones to speaker 330 using tone generating instructions 324. Again processor 138 generates a test record by recording the responses and lack of responses from input interface 134 and once the test is complete provides the test results to display interface 136 and customer service data system 162. At which time the user and the hearing health professional may discuss the user's need of a hearing aid.

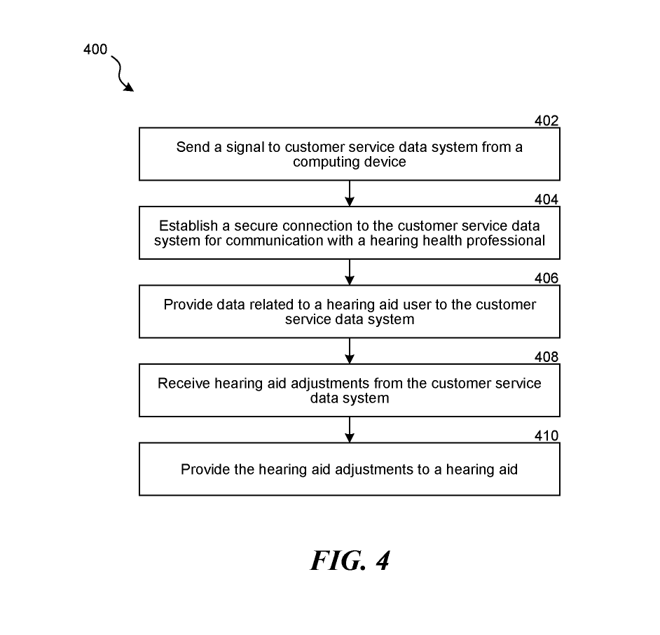

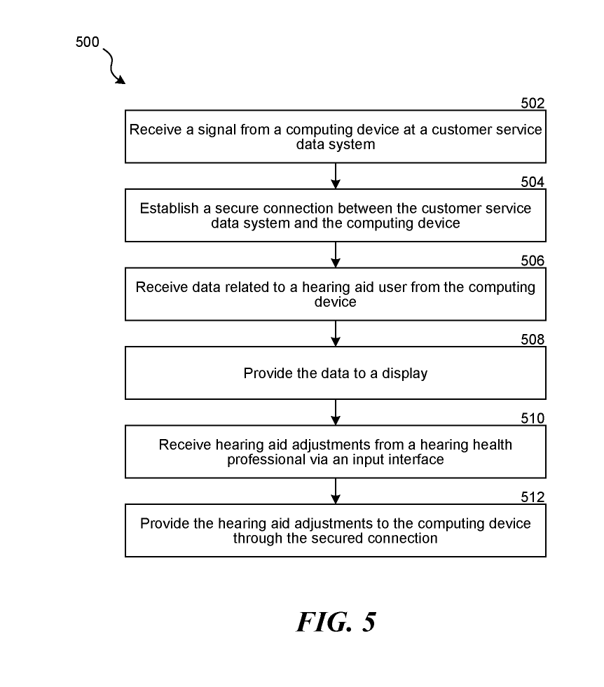

FIGS. 1-3 show systems for providing real-time remote hearing aid adjustment and hearing testing by hearing health professionals. FIGS. 4-5 show methods of preforming real-time remote hearing aid adjustment and testing by hearing health professionals.

FIG. 4 is a flow diagram of an embodiment of a method 400 of receiving hearing aid updates from a customer service data system 162. At 402, computing device 122 sends a signal to customer service data system 162. Advancing to 404, computing device 122 establishes a secure connection to customer service data system 162 for communicating with a hearing health professional. The connection is configured to communicate data and/or audio and/or visual data between computing device 122 and customer service data system 162. Proceeding to 406, computing device 122 provides data related to a hearing aid user to the customer service data system 162. The data may include hearing aid profiles (such as hearing aid profile 106 or hearing aid profiles 128), other hearing aid settings, hearing loss profile, customer ID 133, hearing health professional ID 132, or other data.

Advancing to 408, computing device 122 receives hearing aid adjustments from the customer service data system 162. The hearing aid adjustments may include new hearing aid. profiles, adjusted hearing aid profiles, other hearing aid settings, hearing aid application updates. Proceeding to 410, computing device 122 provides the hearing aid adjustments to hearing aid 102.

FIG. 4 shows a method 400 performed by computing device 122. FIG. 5 shows a method performed by customer service data system 162.

FIG. 5 is a flow diagram of an embodiment of a method 500 of providing hearing aid updates to a hearing aid user. At 502, customer service data system 162 receives a signal form computing device 122. The signal may contain hearing health professional ID 132 and/or customer ID 133 for routing to the correct interface 168 and hearing health professional, Proceeding to 504, customer service data system 162 establishes a secure connection with computing device 506. Advancing to 506, customer service data system 162 receives data related to a hearing aid user. Proceeding to 508, customer service data system 162 provides the data to a display (such as interface 168). Advancing to 510, customer service data system 162 receives hearing aid adjustments form the hearing health professional at an input interface (such as interface 168). Proceeding to 512, customer service data system 162 provides the hearing aid adjustments to computing device 512 through the secured connection.

According to the above referenced drawings embodiments of systems and methods are disclosed including an embodiment of customer service data system configured 162 to initiate a communications link with computing device 122 to communicate data related to a hearing aid setting and receiving the data from computing device 122. In this embodiment, customer service data system 162 provides the data to a display for viewing by a hearing health professional and in response receives adjustments to the hearing aid setting at input interface 168 and provides the hearing aid adjustments to computing device 122 through the communication link.

aspect of the present embodiment of the communication link with computing device 122 is further configured to communicate audio between a hearing aid user and a hearing health professional. In another aspect of the present embodiment, communication link is further configured to communicate visual data between the hearing aid user and the hearing health professional. In yet another aspect of the present embodiment, the data includes the user's hearing loss profile.

In another embedment, computing device 122 establishes a first communications link with customer server data system 162 to communicate data related to a hearing aid setting from a hearing health professional to a hearing aid of a user. Computing device 122 provides the data related to hearing aid settings to customer service data system 162 and in response computing device 122 receives an adjusted hearing aid settings in response to providing the data. Computing device 122 provides the adjusted hearing aid profile to a hearing aid through a second communication link.

In one aspect of the present embodiment, the first communication link allows the user to hold a conversation with the hearing health professional in addition to transferring the data. In another aspect of the present embedment, computing device 122 identifies the hearing health professional who is assigned to the user and establishes the first communication link with a customer service data system (such as customer service system 262) assigned to the hearing health professional. In yet another aspect, the hearing health professional who is assigned to the user is identified by an identifier (such as hearing health professional ID 132) stored on computing device 122. In yet another aspect of the present embodiment, the customer service data system 162 and the assigned to the hearing health professional are identified by an identifier (such as hearing health professional ID 132) stored on the computing device 122. In yet another aspect, computing device 122 provides the user with a user selectable list of a plurality of hearing health professionals and receives a user input corresponding to a selection of one of the plurality of hearing health professionals. In response, computing device 122 establishes the first communication link with the customer service data system assigned to the selected one of the plurality of hearing health professionals. In another aspect of the present invention, computing device 122 provides a user with a user selectable schedule for scheduling an on-line appointment with the hearing health professional and receives data related to a user selection (such as an appointment time). In response to the user selection, computing device 122 transmits the data to customer service data system 162 to schedule an appointment over the communication link. Computing device 122 receives confirmation of the availability of the appointment from customer service data system 162 and provides the appointment to the user via display interface 136.

In another embodiment, computing device 122 comprises a cell phone including a network interface (such as network interface 146) adapted to communicate with a communications network and a transceiver (such as transceiver 144) configured to communicate with hearing aid 102. The cell phone further includes a user interface for displaying information and for receiving user input and processor 138 coupled to network interface 146 and transceiver 144 and to the user interface. A computer-readable storage media (such as computer-readable storage media 124) is coupled to processor 138, computer-readable storage media 124 stores data about the hearing aid including contact data for a hearing health professional that programmed the hearing aid and instructions that, when executed by the processor 138, cause the processor to: (1) provide a graphical user interface (GUI) to the display, the GUI including a user-selectable option for contacting the hearing health professional; (2) receive a user input corresponding to the user-selectable option; and (3) control network interface 146 to establish a communication link with the hearing health professional in response to receiving the user input.

In an aspect of the present embodiment, computer-readable storage media 124 further includes instructions that, when executed, cause the processor 138 to communicate the data about the hearing aid to a remote device associated with the hearing health professional (such as customer service data system 162 or customer service system 262) and receive updated data for updating hearing aid 102 from the remote device. The update data may include, at least one hearing aid profile, software updates, or firmware updates.

In another aspect of the present invention, computer-readable storage media 124 further includes instructions that. When executed, cause processor 138 to communicate at least a portion of the updated data to hearing aid 102 via transceiver 144. In some instances the data may include one or more environmental sound samples. The computer-readable storage media may also include additional instructions that, when executed, cause the processor to: (1) receive a schedule of to the hearing health professional from the communication network, and wherein the GUI includes a user selectable scheduler including one or more elements for reserving an available time slot in the schedule for an appointment with the hearing health professional; (2) receive a user input relating to a time; (3) schedule a time to establish the communication link, and (4) establish the communication link with the hearing health professional at the time of the appointment. In one aspect, the processor also stores an identifier corresponding to the hearing health professional after a communication link is established. In one particular aspect the communication link is a secure voice-data link.

In yet another embodiment, a computing system includes a first transceiver adapted to communication with a communications network and a processor coupled to the transceiver. The computer system may also include a second transceiver configured to communicate with at least one hearing aid. The computing system also includes a computer-readable storage media accessible to the processor, the computer-readable storage media configured to store instructions that, when executed by the processor, cause the processor to: (1) perform a hearing evaluation and (2) conference call a hearing health professional. In one aspect, the hearing evaluation is conducted by the hearing health professional. In another aspect, the hearing evaluation is automated hut the results are provided to the hearing health professional. In one particular aspect, the hearing evaluation includes a hearing screening followed by a hearing test if the hearing screening indicates hearing loss. In one aspect, the hearing evaluation is performed on the hearing aids via the computing device.

In yet another embodiment, a computing system including a transceiver adapted to communicate with a communications network, an output interface configured to provide an audible signal to a speaker, and a user interface for transmitting data to and from a user. The computing device further includes a processor coupled to the transceiver, the output interface, and the input interface. The processor is further coupled to a computer-readable storage media, the computer-readable storage media configured to store instructions that, when executed by the processor, cause the processor to: (1) provide one or more audible tones to the speaker; (2) receive inputs relating to the audible tone from the user interface; (3) generate a hearing test result; and (4) provide the hearing test result to a hearing health professional. In one aspect of the present embodiment, the computing device receives control signals from a server over the communication network. The control signals may include gain and frequency data. In another aspect, the processor when executing the instructions also provides a user-selectable option to the user interface. In one particular aspect, the speaker is a headphone set and in another aspect the speaker is a hearing aid.

Although the present invention has been described with reference to preferred embodiments, workers skilled in the art will recognize that changes may be made in form and detail without departing from the scope of the invention.

What is claimed is:

1. A method for remotely tuning a hearing aid, the method comprising:
receiving, at a computing device, a user input indicative of a selection to provide data related to settings of the hearing aid, wherein the data at least includes a description of a problem with the hearing aid settings and/or a description of a desired adjustment to the hearing aid settings;
transmitting, from the computing device, the data related to the hearing aid settings to a remote customer server system configured to provide data associated with hearing aid tuning adjustments;
receiving, at the computing device, one or more hearing aid tuning adjustments to the hearing aid settings from the remote customer server system, wherein the one or more hearing aid tuning adjustments are based, at least in part, on the data related to the hearing aid settings; and
transmitting, from the computing device, the adjusted hearing aid settings to the hearing aid.

2. The method of claim 1 wherein the hearing aid settings include a hearing aid profile for the user, and wherein the hearing aid profile is configured to be applied by a processor of the hearing aid to process sounds to produce a modulated output signal that compensates for a hearing deficiency of the user.

3. The method of claim 2 wherein receiving the one or more hearing aid tuning adjustments to the bearing aid settings includes receiving an adjusted hearing aid profile, and wherein transmitting the adjusted hearing aid settings includes transmitting the adjusted hearing aid profile to the hearing aid for application by the processor of the hearing aid.

4. The method of claim 3 wherein the adjusted hearing aid profile does not compensate for a particular acoustic environment of the user.

5. The method of claim 3 wherein the adjusted hearing aid profile is further configured to be applied by the processor of the hearing aid to process sounds to produce a modulated output signal that compensates for a particular acoustic environment of the user.

6. The method of claim 1 wherein the data related to the hearing aid settings does not include data about an acoustic environment of the user.

7. The method of claim I wherein the data related to the hearing aid settings includes data about an acoustic environment of the user.

8. The method of claim 1 wherein the data related to the hearing aid settings includes a hearing loss profile for the user.

9. The method of claim 1 wherein the one or more hearing aid tuning adjustments are determined by a hearing health professional.

10. The method. of claim 1 wherein the data related to the hearing aid settings includes only the description of a problem with the hearing aid settings and/or the description of a desired adjustment to the hearing aid settings.

11. The method of claim 1 wherein the computing device is a personal digital assistant (PDA), smart phone, portable computer, tablet computer, or other mobile communication device.

12. A method for remotely tuning settings of a hearing aid, comprising:
receiving, at a server, a user request for tuning of the hearing aid, wherein the user request includes at least a description of a problem with the hearing aid settings and/or a description of a desired adjustment to the hearing aid settings;
transmitting the user request from the server to a remote computing device configured to provide data associated with hearing aid tuning adjustments;
receiving, at the server, one or more hearing aid tuning adjustments to the hearing aid settings from the computing device configured to provide data associated with hearing aid tuning adjustments, wherein the one or more hearing aid tuning adjustments are based, at least in part, on the user request; and
transmitting the one or more hearing aid tuning adjustments to the hearing aid settings from the server to a remote computing device associated with a user of the hearing aid.

13. The method of claim 12 wherein the hearing aid settings include a hearing aid profile for the user, wherein the hearing aid profile is configured to be applied by a processor of the hearing aid to process sounds and produce a modulated output signal that compensates for a hearing deficiency of the user, wherein the one or more hearing aid tuning adjustments include an adjustment to the hearing aid profile, and wherein the adjustment to the hearing aid profile is based on the user request and an individual hearing loss profile for the user.

14. The method of claim 12, further comprising transmitting at least a portion of the hearing aid settings from the server to the computing device configured to provide data associated with hearing aid tuning adjustments.

15. The method of claim 12, further comprising transmitting an individual hearing loss profile for the user from the server to the computing device configured to provide data associated with hearing aid tuning adjustments.

16. The method of claim 12 wherein the user request includes only the description of a problem with the hearing aid settings and/or the description of a desired adjustment to the hearing aid settings.

17. The method of claim 12, further comprising:
establishing a secure connection between the server and the computing device configured to provide data associated with hearing aid tuning adjustments; and
establishing a secure connection between the server and the computing device associated with the user of hearing aid.

18. A system for remote tuning of a hearing aid profile associated with a user, the system comprising:
a network interface configured to communicate with a network;
a processor coupled to the network interface; and
a computer-readable storage media accessible to the processor, the computer-readable storage media configured to store instructions that, when executed by the processor, cause the processor to
receive, via the network, a user request for tuning of the hearing aid profile, wherein the hearing aid profile is based, at least in part, on a hearing loss profile associated with the user, and wherein the user request is generated by an application running on a remote computing device associated with the user;
transmit the user request to a remote computing device configured to provide data associated with hearing aid tuning adjustments;
receive a hearing aid tuning adjustment to the hearing aid profile from the computing device configured to provide data associated with hearing aid tuning adjustments, wherein the hearing aid tuning adjustment is based, at least in part, on the user request; and
transmit, via the network, the hearing aid tuning adjustment to the hearing aid profile to the remote computing device associated with the user.

19. The system of claim 18 Wherein the user request includes a description of a problem with the hearing aid settings and/or a description of a desired adjustment to the hearing aid profile.

20. The system of claim 18 wherein the user request includes the hearing aid profile of the user and/or the hearing loss profile of the user.

21. The system of claim 18 wherein the hearing aid profile is configured to be applied by a processor of a hearing aid to process sounds to produce a modulated output signal that compensates for a hearing deficiency of the user, and wherein the hearing aid tuning adjustment to the hearing aid profile includes a frequency and/or amplitude adjustment that better compensates for the hearing deficiency of the user.

22. The system of claim 18 wherein the instructions, when executed by the processor, further cause the processor to display the user request and/or one or more settings of the hearing aid profile on an interface of the computing device configured to provide data associated with hearing aid tuning adjustments.

23. The system of claim 18 wherein the instructions, when executed by the processor, further cause the processor to transmit, via the network, a communication from a hearing health professional to the remote computing device associated with the user.

24. The system of claim 23 wherein the communication from the hearing health professional includes a text communication associated with the hearing aid tuning adjustment to the hearing aid profile, and wherein the text communication is displayable on the remote computing device associated with the user via the application running on the remote computing device associated with the user.

25. The system of claim 23 wherein the communication from the hearing health professional includes a request for the user to visit the hearing health professional.

26. A mobile-computing system for remote tuning of a hearing aid profile associated with a user, the system comprising:
a network interface configured to communicate with a network;
a processor coupled to the network interface;
an input interface coupled to the processor and configured to receive user input;
a transceiver coupled to the processor and configured to communicate with a hearing aid; and
a computer-readable storage media accessible to the processor, the computer-readable storage media configured to store instructions that, when executed by the processor, cause the processor to
receive, via the input interface, a user input indicative of a selection to provide information related to settings of the hearing aid profile, wherein the hearing aid profile is based, at least in part, on a hearing loss profile associated with the user;
transmit, via the network, the information related to the hearing aid settings to a remote server system configured to provide data associated with hearing aid tuning, adjustments;
receive, from the remote server system via the network, a hearing aid tuning adjustment to the hearing aid profile, wherein the hearing aid tuning adjustment is based, at least in part, on the information related to the hearing aid settings; and
send the hearing aid tuning adjustment to the hearing aid via the transceiver.

27. The system of claim 26 wherein the information related to the hearing aid settings includes a description of a problem with the hearing aid settings and/or a description of a desired adjustment to the hearing aid settings.

28. The system of claim 26 wherein the information related to the hearing aid settings includes only a description of a problem with the hearing aid settings and/or a description of a desired adjustment to the hearing aid settings.

29. The system of claim 26, further comprising:
a display interface for displaying information, and
wherein the instructions, when executed by the processor, further cause the processor to display a communication from a hearing health professional at the display interface, wherein the communication is received from the remote server system via the network, and wherein the communication is associated with the hearing aid tuning adjustment to the hearing aid profile.

30. The system of claim 26 wherein the hearing aid profile is configured to be applied by a processor of the hearing aid to process sounds to produce a modulated output signal that compensates for a hearing deficiency of the user, and wherein the hearing aid profile does not compensate for a particular acoustic environment of the user.
